(19) United States
(12) Patent Application Publication
(10) Pub. No.: US 2005/0051179 A1
(43) Pub. Date: Mar. 10, 2005

(54) Simple apparatus for preparing experimental models of brain neuronal damage and preparation of secondary brain damage model with methods for evaluation of drug efficacy of novel compounds against brain damage and memory-related events

(76) Inventors: Foo Wah Foong, Kyoto City (JP); Masato Kuwabara, Yokohama City (JP)

Correspondence Address: Beck & Tysver, P.L.L.C., Suite 100, 2900 Thomas Avenue S., Minneapolis, MN, 55416, US

(21) Appl. No.: 10/657,576

(22) Filed: Sep. 8, 2003

(51) Int. Cl. A61B 19/00; A61K 49/00

(52) U.S. Cl. 128/897; 128/898; 119/417; 424/9.2

(57) ABSTRACT

An apparatus inflicts brain damage of secondary nature, including neuronal and cerebral vascular damage, in a controlled and reproducible manner in animals for experimentation. The apparatus includes a barrel or delivery channel through which a weight is dropped under gravitational force. The weight is dropped from a controlled height to thereby control the force with which the weight impacts the animal's exposed brain site and thereby controls the degree of brain damage inflicted. Effects of drugs on the post-impact brain damages can be evaluated by magnetic resonance imaging (MRI) and/or MRI-based blood flow in a time-related manner. The experimental model injures memory-related brain regions to induce memory loss, and evaluation of memory-related effects of drugs can also be performed using an innovative maze.

### FIELD OF THE INVENTION

The present invention relates to an apparatus for the preparation of an experimental model for brain damage comprising neuronal and vascular impairments and evaluation for drug efficacy against brain impairments/damage and memory-related learning with magnetic resonance imaging (MRI) and a memory-related maze. Briefly, a simple and reliable device that reproducibly inflicts cerebral injuries in experimental animals such as the rat, cat and dog. Brain damage, including secondary brain damage (SBD), is evaluated on a time-related basis with MRI and/or MRI-based cerebrovascular flowmetry whereupon these parameters serve as analytical indexes of drug efficacy for SBD and memory-associated learning via MRI and/or behavioral responses.

### BACKGROUND OF THE INVENTION

Brain damage due to traumatic impacts includes ischemia, edema and enhanced intracranial pressure etc. to exacerbate brain tissue/neuronal damage, leading to SBD. The important therapies against traumatic impacts involve prevention of SBD development. Current experimental models can not provide avenues for evaluation of drug efficacy of novel chemical agents for preventing SBD development. The hitherto extensively practiced method of inflicting brain damage mainly involves occlusion-reperfusion of the common carotid artery and induces abnormal cerebrovascular flow (CBF), rendering studies on SBD prevention incomplete. In addition, repeating the occlusion-reperfusion procedure is highly invasive and requires intensive training and practice. Although the occlusion-reperfusion model may serve as an approach to elucidating the mechanism of cerebral injuries and evaluating drug efficacy, the actual state of the brain after external impacts and subsequent events such as delayed cerebral damage and SBD cannot be established with such prior methods.

Development of an experimental model of head injury includes neurobehavioral measurements of outcome after impact or damage inflicted on the head of animals, and deficits involving reflexes have been the focal measure to assess the validity of previous experimental models (Chen et al., 1997; Mesenge et al., 1996; Tang et al., 1997; Hannay et al., 1999). However, very often damage inflicted on animals by previous methods was either fatal or too severe to perform studies for subsequent evaluation. Typical of such outcomes is the graded severity impacted by a rod producing mild and/or significant contusion at the impact site with neuronal damage in mice (Hannay et al., 1999) using a 3-mm-diameter impactor rod adjusted at an angle 45 degrees to the vertical and with curved tip of said impactor rod slamming perpendicular to the craniectomized brain surface (Cherian et al., 1998). Although Yoshino et al. (1982) have introduced a more objective approach in analyzing brain damage of head injuries using MRI, studies on an appropriate experimental model with such an objective approach has yet to be attempted. Compression of air can generate a force which in turn delivers a pellet to any target surface. Such method is expensive and post-impact conditions of brain damage may not be reproducible. Furthermore, the types of brain damage cannot be easily adjusted to furnish experiments specific for certain investigations or evaluation of certain chemical agents for certain damage, facilitating drug evaluation to prevent and/or treat SBD or post-trauma brain injuries.

### SUMMARY OF THE INVENTION

The present invention is directed to an apparatus and method for reproducibly establishing secondary brain damage. The object of this apparatus and method is to mimic the type of brain damage that is caused by accidental head injury. Treatments for such brain damage can then be studied using animals in which secondary brain damage has been inflicted via the apparatus and method of this invention.

To achieve reproducible brain damage, a weight is dropped onto the exposed brain site of an animal. The weight and the distance the weight should travel to achieve the desired level of brain damage depends upon the species of the animal.

An analytical approach of using behavioral, MRI and cerebrovascular flowmetry is used in assessing the SBD-related outcome, followed by behavioral studies involving memory-associated learning using a maze before pathological studies were performed to confirm neuronal damage in memory-related brain sites.

However, our present invention furnishes a model where an actual clinical state of external trauma due to impact can be demonstrated. The present invention does not employ compressed air to force an object to impact on the brain.

According to one aspect of this invention, an apparatus is used to inflict brain injury for preparing experimental models of SBD comprising neuronal and cerebral vascular impairments including damage of memory-related neurons/tissues in the brain.

The injury-inducing apparatus has a barrel or delivery channel through which a weight travels. In use, the barrel is positioned to run generally vertically and the weight travels therethrough under gravitational force. The barrel is sized to accommodate the weight and to fit well into a craniectomized brain site. The apparatus further includes a pin or stop member extending through the delivery channel or barrel. This pin supports the weight until the weight is selectively delivered. When the pin is removed, the weight drops through the delivery channel and onto the exposed brain site to inflict SBD, contusion, concussion and other relevant cerebral injuries.

In the present invention, the barrel includes a rigid and hollow cylindrical rod having a linear configuration. The linear configuration has an outer diameter sized such that at least one of the two opened ends of the barrel fits into the craniectomized cavity of the skull when positioned upright. The distally opposite open end of the barrel allows a drop-weight (DW) means to be accommodated, with the drop-weight supported by a pin or stop member before dropping onto the exposed brain surface at will by guiding movement of the weight member through the barrel or delivery channel. The barrel is of variable length or the path traveled by the weight through the barrel is variable and selectable. The barrel is formed of a rigid material. At variable intervals of the barrel are bilaterally drilled pin-holes for accommodating a pin member or like stop member which extends through aligned pin-holes to support the drop-weight until the operator chooses to drop the weight by removing the supporting pin.

The weight includes a rigid solid cylindrical rod having a linear configuration of uniform radius of curvature that is sized to fit well within the barrel. The weight has a radius of curvature slightly smaller than and length shorter than the barrel to be guided by the barrel in impacting a target site of the brain. The weight has a smooth flat surface with right-angled edges and weight appropriate for a particular animal species being used in preparing the SBD. The end distal to the impacting end has a connection member with a wire or thread material to the end of the pin member for easy retrieval of the weight immediately after impact.

The pin member has a linear configuration of a radius curvature fitting loosely across the bilaterally drilled pin-holes in the barrel member with the distal end attached to the dropping weight distal end by a wire or thread to facilitate prompt recovery of the weight member after impact. The pin member is made of a rigid material.

An impact of 10-250 cm·g delivered to a surface area of about 19.63 mm² causes cerebral damage in rats. An impact of 45-55 cm·g delivered to a surface area of about 19.63 mm² causes incidences of SBD in rats. An impact of 10-480 cm·g delivered to a surface area of about 19.63 mm² causes cerebral damage in cats and dogs. An impact of 200-280 cm·g delivered to a surface area of about 19.63 mm² causes incidences of SBD in cats and dogs. A surface area of about 19.63 mm² results, for example, by using a weight having an impact end that is circular in cross section having a diameter of about 5 mm. Of course, other geometries would be suitable as well.

According to another aspect of the invention, a method for inflicting brain damage in a reproducible manner is described. The method includes exposing a portion of the surface of the brain via craniectomy, positioning an open end of a delivery channel or barrel immediately adjacent the exposed brain surface, delivering a weight through the barrel under gravitational force onto the brain surface. The weight of the weight, its surface area where it contacts the brain and the height from which it is dropped determine the impact on the brain, and these parameters are controlled or selected to generate the desired degree of brain damage. These parameters vary by species.

A memory-evaluation maze is used to measure the effects of the inflicted brain damage. The maze is constructed to allow experimental animals to move freely in horizontal directions with visible planar views. Animals are allowed to move freely within a certain open space that connects to another open space invisible to the animals through at least four channels where animal feed as a reward is placed at the end of at least one of the channels. Animals are trained to remember the path that leads to a reward, i.e., feed availability at a certain path for all animals.

Evaluations of novel chemical agents on the inflicted SBD and relevant post-impact physiological and pathological events are conducted using MRI, cerebral blood flowmetry and/or behavioral analyses. SBD is generated by dropping the weight according to the species-specified dropping distance and weight onto a unilaterally craniectomized brain surface without removing the dura mater before/after drug administration for evaluation purposes. Analysis of memory-related events is evaluated with a self-designed maze before and after impact with use of either drug or drugs.

The present invention furnishes a non-invasive experimental model for drug evaluation on cerebral injuries such as SBD generated according to weight and distance for dropping the weight for a certain species to generate an impact. The present invention further provides an assessment method for evaluation of novel chemical agents related with memory.

Furthermore, the present invention provides an avenue for drug evaluation on cerebral injuries after impacting the weight on a unilateral cerebral site without invasive procedures and according to species specifications on impact force using magnetic resonance imaging (MRI), a non-invasive time-related evaluation approach in assessing post-impact events.

The present invention also provides another approach to drug evaluation using a non-invasive species-specified impact to generate cerebral injuries and to assess time-related memory events.

In an alternative embodiment of the present invention, the end of barrel member that fits into the target impact-site has an outer diameter that is less than the diameter of adjacent portion of the barrel member.

It is an object of the present invention to provide a simple apparatus that enables investigation personnel to prepare an experimental model for studying post-impact events of SBD in the physiological and pathological aspects for innovating therapies and developing of novel chemical agents, accordingly.

It is also an object of the invention to establish a reliable and reproducible experimental model for evaluation of drug efficacy on SBD and memory-related aspects through a simple approach in testing the efficacy of chemical agents for post-impact treatment and memory-relevant perspectives.

It is a further object to provide the design of a simple maze to evaluate drug efficacy of novel chemical agents for alleviation of memory-related deficits through a simple yet reliable experimental model that would save time, effort and material inputs compared with routine memory tests that are very much defeated by the perfect state of brain, which in actual state is somewhat pathological.

These and other objectives and advantages of the present invention will become apparent from a reading of the attached specification and appended claims.

The present invention relates to an experimental model for cerebral neuroimpairments using experimental animals susceptible to brain injuries. One end of a rod with cylindrical configuration where both ends are opened is affixed to the exposed cerebral region of brain while the opposite open-end allows insertion of the weight to be positioned at various species-specified distances and guide dropping of the drop-weight to impact on the exposed brain are special features of the invention.

The invention can induce concussion, contusion and SBD as desired. For rats, a weight of cylindrical configuration with a diameter of 5 mm is used to generate an impact force of 45-55 cm·g on the skull to inflict SBD. For cats and dogs, a weight of cylindrical configuration with a diameter of 3-15 mm is used to obtain an impact force of 200-280 cm·g on the skull to inflict the SBD.

A maze for evaluation of memory derived from the invention allows experimental animals to move freely in horizontal directions with visible planar views within a certain open space that connects to another channel-partitioned open space invisible to the animals through not less than four such channels and the animals locate feed as a reward that is placed on a feed lot at the end of the channels.

One aspect of the invention serves as an evaluation method for drug efficacy on brain injuries. After drug administration, one open end of the barrel is affixed onto surgically exposed brain surface of unilateral hemisphere of experimental animal without removal of dura mater. A weight is dropped from a predetermined, species-specific drop-distance/height and falls onto the exposed brain surface to cause an impact. The post-impact brain injuries are monitored by MRI diagnosis of the cerebral injury.

Another aspect of the invention serves as an evaluation method for drug efficacy on brain injuries. After drug administration, one open end of the barrel is affixed onto the surgically exposed brain surface of a unilateral hemisphere of the experimental animal without removal of dura mater. A weight is dropped from a predetermined, species-specified drop-distance/height and falls onto the exposed brain surface to cause an impact. Post-impact brain injury is evaluated by timing behavior.

Still another aspect of the invention serves as an evaluation method for drug efficacy on brain injuries. One open end of the barrel is affixed onto the surgically exposed brain surface of a unilateral hemisphere of the experimental animal without removal of dura mater. After treatment with test compound, a weight is dropped from a species-specified drop-distance/height and falls onto the exposed exposed brain surface to cause an impact, whereupon time-related post-impact brain damage is monitored by MRI portrayals.

Yet another aspect of the invention serves as an evaluation method for drug efficacy on brain injuries at memory sites. One open end of the barrel is affixed onto the surgically exposed brain surface of a unilateral hemisphere of an experimental animal without removal of dura mater. After treatment with a test compound, the weight is dropped from a species-specified drop-distance/height and drops onto the exposed brain surface to cause an impact, whereupon time-related brain damage in relation to memory evaluation is monitored by the maze.

### BRIEF DESCRIPTION OF THE DRAWINGS

FIG. 1 is an exploded perspective view of an apparatus for inflicting brain injury in a controlled manner;

FIG. 1a is a side-elevational view of the apparatus of the present invention positioned for use with a rat;

FIG. 2 is a perspective view of the memory evaluation maze, used in conjunction with a method for evaluating brain injury and brain response to chemical agents.

FIG. 3 is a top view of the memory evaluation maze of FIG. 2.

FIG. 4 illustrates MRI findings (T2-weighted images) of post-impact brains of non-treated (a) and treated (b) groups at 48 hr after impact with the invention.

FIG. 5 is a graph showing histopathological evaluation of changes in the rate of brain-surface damage area after impact with the weight-drop method of the present invention in rats without (a) and with (b) drug pretreatment.

FIG. 6 portrays changes in a brain damaged area (CA3) at 48 hr after impact with the weight-drop method of the present invention in rats without (a) and with (b) drug pretreatment from FIG. 5 with MRI application.

FIG. 7 is a graph showing changes in viable pyramidal cell counts per unit area of hippocampal CA1 field after impact with the weight drop method of the present invention in rats without (a) and with (b) drug pretreatment.

FIG. 8 is a graph illustrating changes in viable pyramidal cell counts per unit area of hippocampal CA3 field after impact with the weight drop method of the present invention in rats without (a) and with (b) drug pretreatment.

FIG. 9 represents typical MRI findings of dogs before (a) and those subjected after 18 hr (b) and 24 hr (c) to direct contusion nest (DCN).

FIG. 10 represents typical MRI portrayals of dogs at 0 (a) immediately after) and (b) 3 hours after impact with the drop-weight device of the present invention.

FIG. 11 is a graph showing time-related changes in cerebral blood flow (CBF) velocity of non-treated (broken line) and MK-801-treated (solid line) dogs inflicted with direct contusion nest (DCN).

FIG. 12 is a graph showing time-related changes in cerebral blood flow (CBF) velocity of non-treated (broken line) and MK-801-treated (solid line) dogs inflicted with the drop-weight apparatus of the present invention.

### DETAILED DESCRIPTION OF PREFERRED EMBODIMENT(S)

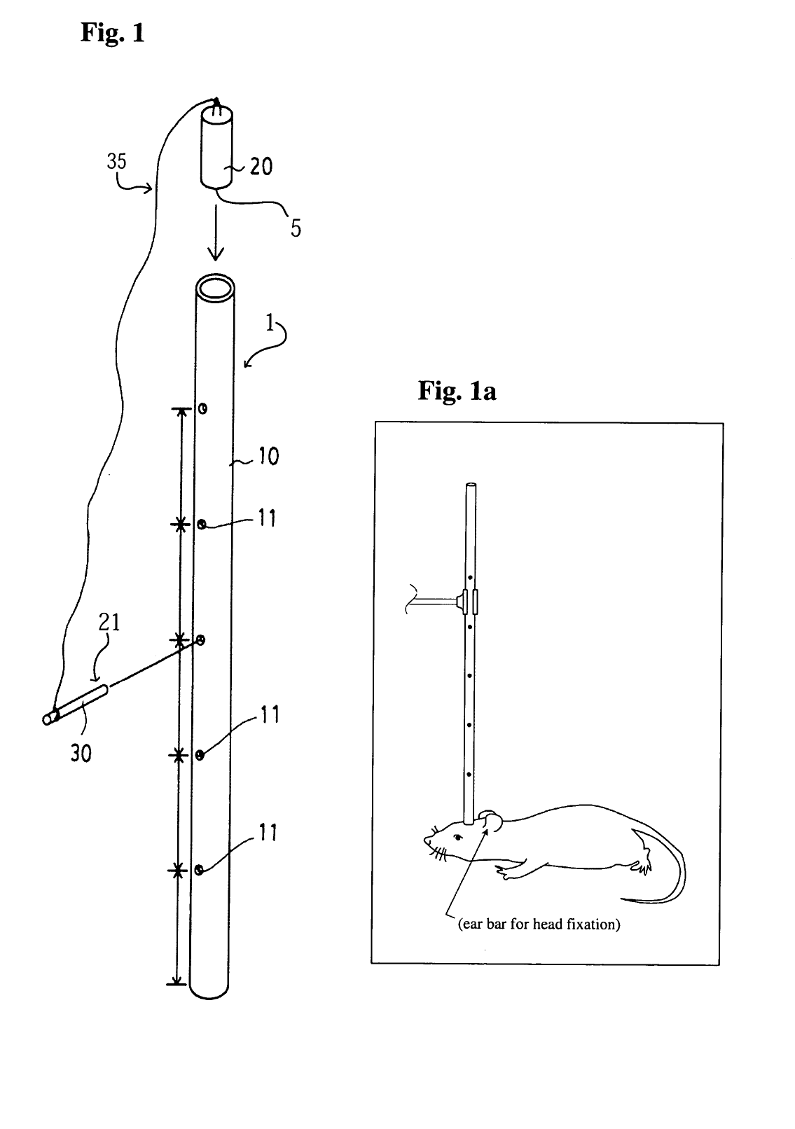

FIG. 1 illustrates a drop weight apparatus for inflicting brain injury in a controlled manner. The drop weight apparatus 1 comprises a guide barrel or delivery channel 10, having a generally cylindrical internal diameter. A weight 20 is placed inside the barrel 10 and kept in position at a specified distance inside barrel 10 prior to dropping the weight 20 to generate an impact on an exposed brain surface with dura mater intact by surgery/drilling. In this manner, injury is inflicted in an essentially non-invasive way; that is, the brain itself is not surgically compromised, though the skull is opened to expose the brain. Appropriate dimensions of barrel 10 and weight 20 are determined by the species upon which the apparatus 1 is used. The barrel 10 and weight 20 can be made of any material that can perform the functions described herein. Examples of materials that may be suitable include: plastic, paper, steel, iron, bronze and stainless steel. Stainless steel offers advantages in sturdiness and durability. The impact end 5 of weight 20 preferably is cylindrical terminating in a flat cross-sectional surface for good contact with brain surface upon impact.

To avoid resistance being generated between barrel 10 and weight 20 on dropping, the dimensions of the barrel and weight are specified to generate the required impact to inflict desired cerebral injuries. With negligible experimental errors, the dimensions of barrel 10 and weight 20 are specified with values that afford least resistance with least contact movement between the inner surface of barrel 10 and outer surface of weight 20, such that the impact is perpendicularly generated rather than impacting with a non-uniform force on the brain surface.

The present invention serves as an experimental model to inflict cerebral injuries plausible by delivery of an impact through dropping weight 20 with specified/fixed mass from a specified distance under gravitational force. The weight 20 therefore varies according to the animal species employed for the experiment model. Experimental animals appropriated for the present invention include cats, dogs and rats. Guide-barrel 10 has a length ranging from 25 to 35 cm, and this dimension may be adjusted according to the animal species.

The apparatus 1 includes a weight restraining mechanism 21. A preferred embodiment of the weight restraining mechanism 21 involves a pin or stop member 30 extending across the internal diameter of the barrel 10 to support the weight thereon. More specifically, the wall of barrel 10 has one or more pin holes 11 extending laterally therethrough. Preferably, pin holes 11 extend through opposite walls of the barrel 10; in other words, pin holes 11 extend through barrel walls at opposite ends of a diameter line, such that two pin holes 11 are diametrically opposed or laterally aligned. Preferably, several pin hole locations 11 are spaced along the vertical length of the barrel 10 at regular intervals and are used selectively to drop the weight 20 from a selected height as will be described below.

The pin holes 11 receive a pin member or stop member 30. Pin 30, extending through pin holes 11 and supported by the portion of the barrel defining the pin holes 11, supports the weight 20 temporarily in barrel 10. That is, in use, the pin 30 is placed through the selected pin holes 11; when the weight 20 is dropped into the top of the barrel 10, the weight will fall only to the pin 30 and be stopped thereby. It is important to control the height from which the weight is dropped, such as by using the stop member 30, because this controls the force generated from the weight when it falls onto the animal's exposed brain and this force, in turn, determines the degree of brain injury that will be achieved. The force generated under gravitational influence required to achieve the desired degree of brain damage varies by species.

As one embodiment of the weight restraining mechanism, a tether 35 or string, thread, wire, or the like, of fixed length is connected to weight 20. The tether 35 allows retrieval of weight 20 immediately after impact by pulling the thread/wire such that weight 20 would not be in contact with the brain surface for longer than immediate impact, thus avoiding inconsistent damage to and/or post-impact effects on the brain. In addition, the tether 35 facilitates experimental procedures to complement the whole set-up as a simple, practical and reliable method in inducing an impact on the brain surface to cause reproducible cerebral injuries. The state of connection between pin 30 to weight 20 by the tether 35 in FIG. 1 does not determine the drop-distance but merely serves as a simple and efficient means of retrieving weight 20 after impact besides consistently affording weight 20 and pin 30 in good/efficient keeping.

In a method of inflicting desired brain damage in a controlled and reproducible manner, the brain of an experimental animal is exposed via craniectomy. A pin 30 is inserted through pin holes 11 in the barrel 10 of the weight drop apparatus 1 prior to insertion of weight 20 in barrel 10. Barrel 10 is vertically positioned with a clamp and stand, FIG. 1a, with its lower open end fitted into the exposed brain site such that the open end is in direct contact with the brain surface without causing any compression or pressure on the brain surface in all animal species used. The weight 20 is positioned temporarily by the sustaining pin 30 at a specified distance. As an alternative approach, barrel 10 may be first positioned upright in a vertical position with its lower open end in direct contact with the brain surface before insertion of weight 20 in barrel 10.

On removal of pin 30 from pin-holes 11, the weight 20 drops from the specified height or through the specified distance under gravitational force to impact on the exposed brain surface, resulting in cerebral injury such as contusion, concussion and SBD. This apparatus 1 causes cerebral injury of a consistent pattern given a fixed mass of weight 20 and a specified drop-distance. In short, consistent and reproducible cerebral injury are always produced under stipulated conditions. Because of the impact on the dura mater, incidence of cerebral injuries such as SBD are reproducibly generated when the specified conditions are designated. The present invention therefore provides a simple and reliable device and method to reproducibly generate cerebral injuries for studies.

Impact on brain is determined by the mass of weight 20 and drop-distance/height under gravity. Impact on the brain surface is represented by the product (cm·g) of weight 20 (g) and drop-distance (cm; distance from the lower impact-tip of weight 20 to brain surface). For different animal species, the same force would cause different cerebral injury. Further, the area over which the impact is delivered affects the degree of cerebral injury. For a weight having a circular cross section and having a 5 mm diameter (i.e. an area of roughly 19.625 mm²), the following impacts generate concussion, contusion and SBD in rats: impact is 10-250 cm·g, preferably 15-200 cm·g with further specific emphasis from 20-150 cm·g, especially the impact of range of 45-55 cm·g is appropriate for SBD production and memory disturbance. In addition, impacts for cats and dogs range from 10-480 cm·g, preferably 100-360 cm·g with further specific emphasis from 20-300 cm·g, especially impact within 220-260 cm·g is appropriated for SBD generation and memory disturbance.

This experimental model can be used to evaluate drug efficacy of novel compounds against various cerebral impairments, especially for head injuries. Using experimental animals, novel chemical agents can be tested by administration to the animals before cerebral injuries by impact. Evaluation of improvements of cerebral injuries can also be monitored thereafter by comparison with non-treated animals. As an evaluation method, unless stated otherwise, MRI diagnosis is used. MRI evaluation can quantify damage by applying the imaging approach. The MRI approach can evaluate drug efficacy in a time-related fashion, and affords prevention and assessment of attenuation of SBD development, facilitating development of novel therapeutics for useful prevention and positive treatment of brain injuries, especially SBD. Reliability of evaluation is further enhanced when SBD is induced under specific conditions appropriate for generation of SBD in a reproducible manner. Furthermore, MRI diagnosis enables prompt monitoring of brain damage within about one hour after injury. In other words, animals yielding no or incomplete SBD can be readily identified and omitted from subsequent studies, saving time and unnecessary effort on something inappropriate for evaluation.

In head injuries, this invention impacts on the brain surface to induce cerebral injuries affecting memory-related events after impact. Evaluation of brain injuries can be monitored by MRI diagnosis. Memory based on CA1 and CA3 fields of the hippocampus is affected as MRI portrayals revealed damage at the brain sites after impact.

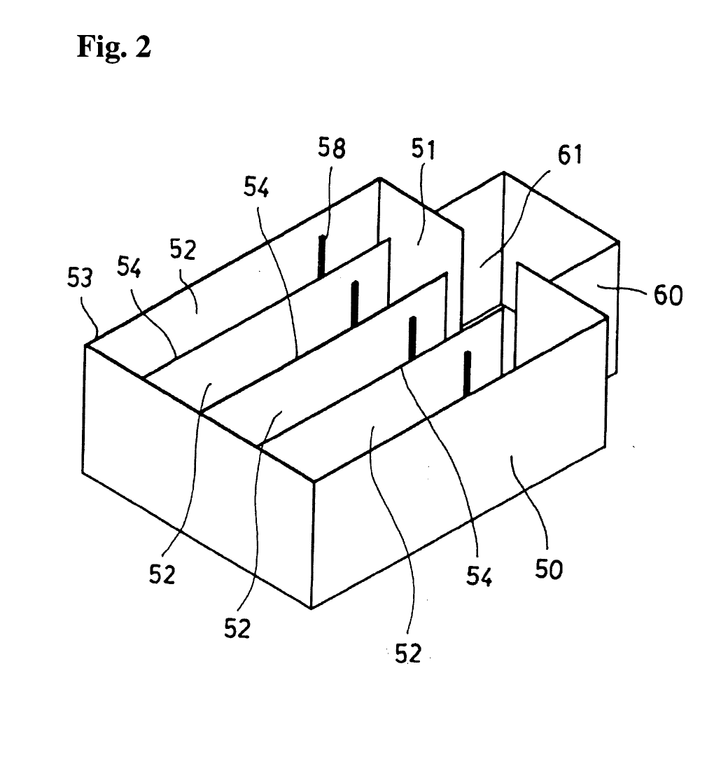

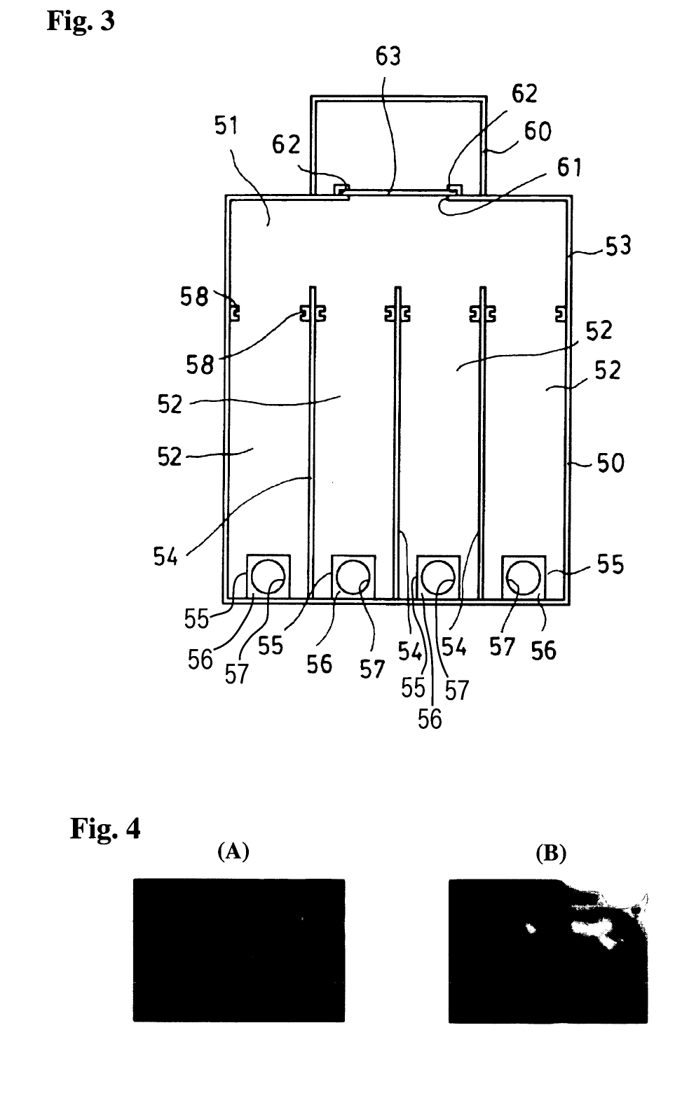

FIGS. 2 and 3 show a maze 50 devised to evaluate memory. The maze accommodates an animal in compartment 60. Maze 50 allows animals to move freely with planar visual view within moving space 51 leading to separate channels 52 with closed ends. The channels 52 are partitioned by walls 54 from adjacent channels, although animals can move freely from space 51 to all channels 52. A preferred maze 50 has four separate channels 52, although additional channels, such as six to eight, may be used. With fewer channels, coincidence may explain the animal's memory test results, rather than being a true indicator of memory. The color, material and size (partition height, width, total length, etc.) of each channel 52 are similar for all channels. The plan view of maze 50 illustrates three partitions 54 installed inside of whole compartment 53 with separate channels 52 aligned in a parallel manner, although a space can be allotted in between two channels 52.

Each closed end of channel 52 has a feed station 55 for feed placement but not within visibility of animals. As shown in FIG. 3, a feed station 55 includes a feeding receptacle 56 in a recess 57. Although not illustrated in the figure, feed station 55 may be designed as a box or trough with surrounding walls. Each inlet of channels 52 has two slots 58 on each side to regulate movement of animals by opening and closure of channels with a wooden/metallic screen that is selectively placed within slots 58 as desired.

Maze 50 is cleansed thoroughly after and before use with antiseptic and chemical agents such as ethanol to deodorize the device. Maze 50 is made of metallic material, and to minimize visual effects on animal memory, inside walls of compartment 53, partitions 54 and channels 52 floor are painted with a dull color, generally black.

As an additional component to maze 50, compartment 60 is installed next to the freely moving space 51 for accommodating a single animal. Door 61 is installed between space 51 and compartment 60. Compartment 60 does not have a specified structure, and it may be a rectangular, square, eliptical or any shape on plan view so long as compartment 60 allows free access of animals to space 51. Movement through door 61 is regulated by a screen 63 where both lateral edges of screen 61 can be slide-fitted into two grooves on lateral sides of door 61 as and when the investigation personnel deem appropriate.

Memory is measured as follows: After cleansing inner component walls of maze 50 with antiseptic ethanol etc. a rat is placed in compartment 60 with door 61 closed, while a pellet of food is placed in recess 57 of feed lot 55. On removal of screen 63 of door 61, the animal searches for food by exploring all channels 52 until the food pellet or reward is found. In this manner, the time that elapses between the removal of screen 63 and the animal locating the reward is measured. The search-reward cycle is repeated until a consistent time interval is established. For all animals, the feed is always placed on the feed lot 55 of the same channel 52. As such, memory of animals can be measured by merely determining the search-reward time interval. Memory can be acquired through repeated learning. As such, animals are required to learn before memory can be tested. In concrete terms, one week before initiation of memory test, animals are not fed for 24 hours, followed by one session daily on the search-reward learning. Results can be represented in such a manner that a certain animal scored a 30-minute interval in the search-reward cycle on the first trial and still presented the same 30-minute interval three to four days after initiation of learning, rendering the search-reward time-interval to stabilize at about 30 days with no less or more improvement thereafter. The time interval is said to have stabilized.

The present invention can be used for evaluation of drug efficacy of novel compounds on cerebral injuries or memory of animals. For example, when animals have been trained prior to head impact, the search-reward time-interval using the maze can be employed to test the drug effects on memory after head impact. If memory was intact, the search-reward time-interval would not be significantly abbreviated. However, if memory was affected, the time interval would be significantly extended. After a certain period has elapsed after head impact, animals are tested with the search-reward time-interval as the index of memory retention/loss. Generally, external head injuries will not affect memory and memory will be reversibly recovered in animals after a period has elapsed after head impact. However, in cases where SBD has been inflicted, memory is affected or delayed (that is, the search-reward time-interval will be extended) and the memory loss is irreversible (not able to locate feed). As such, the innovative maze facilitates and contributes to evaluation of drug efficacy on memory-related events of animals subjected to cerebral injuries by the apparatus and method of the present invention.

Furthermore, regardless of the use of the maze, MRI diagnosis of post-impact cerebral injuries to confirm generation of SBD is preferred. By this confirmation, time and effort can be saved and reliability of drug evaluation on cerebral injuries can be enhanced. In addition in cases where histopathological evaluation has been done, studies on memory recovery of the animals cannot be assessed thereafter, although histopathological evaluation in combination with MRI diagnosis of post-impact cerebral injuries can to a certain extent measure memory outcome without anatomical procedures.

In this manner, various cerebral injuries, especially SBD, can be effected in a simple and reliable fashion with the present invention of inflicting an experimental model of brain damage by a simple and reliable device. In addition, the experimental model thus innovated (experimental animals) can beneficially be employed in the development of novel prophylactics and therapeutics as well as evaluation of drug efficacy of such compounds for cerebral injuries, especially SBD. In particular, use of an MRI diagnosis and maze for memory evaluation affords monitoring/measurements of time-related brain tissue injury which in turn can concretely follow-up on post-impact brain damage. Furthermore, the present invention affords an experimental model for appropriation of MRI application, leading to reliable prediction on imminent SBD.

### EXPERIMENTS

Based on the following experiments performed with the present invention, details are described. The present invention employed in the following experiments is otherwise not restricted, and alterations of the experiments and conditions can be done within the range of not affecting the technical concept of the invention.

### Experiment 1

Construction of Component Apparatus for the Simple and Reliable Experimental Model of SBD

The drop weight apparatus for rats involves a stainless steel barrel 10 with respective inner and outer diameters of 3.5 and 4.0 mm and length 26 cm. From one (lower) open-end (standard end), five supporting sets of pin-holes 11 of 2-mm diameter are drilled across the barrel at 5-cm intervals such that two pin holes are diametrically opposed at each vertical location. Stainless steel pins of 1-mm diameter and 2-cm length serve as supporting pins and are threaded across the two pin-holes in a horizontal position to support the weight. The weight 20 is a stainless steel rod of 3-mm diameter with 7.5-cm length weighing 5 g in mass.

In addition, drop weight apparatus for cats and dogs are made of stainless steel with inner diameter and outer diameter of the barrel 10 respectively measuring 5.5 and 6 mm. The barrel is 30 cm in length. From one open end (standard end), five supporting sets of pin-holes of 2-mm diameter are drilled across the barrel at 5-cm intervals such that two pin-holes are diametrically opposed at each vertical location. The weight 20 is a stainless steel rod of 5-mm diameter with approximately 6.5-cm length and weighing 12 grams. Supporting pins are made of material and dimensions similar to those used for rats.

Preparation of Experimental Model

Rats, cats and dogs were anesthetized and the unilateral parietal bone of skull was craniectomized before surgically exposing the brain surface without removal of dura mater. The (lower) standard end of the barrel 10 was in direct contact with the exposed brain surface without causing any compression. The weight 20 was dropped from a specified distance appropriate for the respective animal species. Table 1 illustrates the relationship between the type of cerebral injuries that resulted from the given weight and drop-distance. Accordingly, under specified conditions, incidences and types of cerebral injuries for rats, cats and dogs can be reproducibly generated using the described drop weight apparatus and following this drop weight method.

TABLE 1
Relationship between types of cerebral injuries and experimental
conditions for different animal species.

| | | Drop-distance (from standard end in cm) | | | | |
|---|---|---|---|---|---|---|
| Species | Weight (g) | 5 | 10 | 15 | 20 | 25 |
| Rat | 5 | CC | SBD* | CT | CT | CT |
| Cat/dog | 12 | CC | CC | CC | SBD** | CT |
| Cat/dog | 15 | CT | CT | CT | CT | CT |

CC: concussion
CT: contusion
SBD: secondary brain damage
*50 cm · g
**240 cm · g

### Experiment 2

Cytoprotective agents were employed against cytotoxicity induced by excessive extracellular glutamate. SBD derived from experimental head injury was confirmed and the pathological state was evaluated by histopathological approach and MRI diagnosis to follow up on the therapeutic effects of such agents. Of the neurochemical changes induced by delayed tissue injury, excessive release of the excitatory neurotransmitter amino acid glutamate in extracellular spaces induced cytotoxicity and abnormalities of intracellular enzymatic systems, causing eventual cell damage—a major induction mechanism responsible for SBD formation.

Experimental Methods

Male Fisher rats weighing 400-450 g were anesthetized with sodium pentobarbital (Nembutal; Dainippon Pharmaceutical Co., Ltd., Osaka) by i.p. administration (40 mg/kg) before the head was restrained in a stereotaxic apparatus. The apparatus described in Experiment 1 was then employed with the exposed brain surface in alignment as shown in FIG. 1a. Weight 20 of 5-gram mass positioned at a drop-distance of 10 cm was dropped onto the brain surface under gravitational force to generate an impact of 50 cm·g, causing a head injury. Follow-up studies of the progress of subsequent events from 1 to 48 hours post-impact were monitored with the MRI approach.

Rats were divided into two groups and treatments were conducted before impact: Group I was not treated with any agent; while Group II was treated with a selective non-competitive N-methyl-D-aspartate (NMDA) receptor antagonist, MK-801 (Tocris Co., Ltd.), at 3 mg/kg (i.p.). Immediately after impact, MRI diagnoses were conducted to categorize animals as (i) no injuries, (ii) no subarachinoid hemorrhage and (iii) external wounds and hemorrhages inflicted on brain surface, etc. Animals having such criteria were omitted from subsequent studies. Animals with confirmed cerebral injuries but without these criteria were divided into groups of six each.

MRI Portrayal

Employing a self-made small QD coil of 8-cm diameter, MRI of superconductor magnetism of magnetic field intensity 0.5T (Flexart MRT-50GP, Toshiba Medical Co., Ltd.) was performed on rats anesthetized with isoflurane (Fluren; Dainippon Pharm. Co., Ltd.). Rats were placed prostrate inside the coil and T1-weighted imaging with SpinEcho (SE) method using T2-weighted imaging with FastSpinEcho (FSE) method under T1-weighted imaging with TR/TE=50/14 and T2-weighted imaging with TR/TE=4000/102 portrayed immediate post-impact events followed by subsequent events from 1 to 48 hours post-impact.

Histopathological Analysis

Along with MRI findings from 1 to 48 hours post-impact, brain injuries were also evaluated histopathologically using the cardioperfusion method with 200 mL of physiological saline (0.9 w/w %) to first remove blood cells followed by fixation with 50 mL of 0.1 MPBS (pH: 7.4) containing 4% paraformaldehyde (PFA). Brain tissues were then embedded in paraffin and coronal sections of the brain specimens were sectioned before staining with the hematoxylin-eosin (H&E) reagent.

With regard to the therapeutic effects of chemical agents tested, viable pyramidal cell counts per unit area (1 mm²) of tissues in hippocampal CA1 and CA3 fields were determined to derive the area of tissue damage/injury using image-analysis software (NIH Co., Ltd.; Image Ver. 1.55). As for the extent of tissue damage, slices with the maximum damage area between two groups were compared.

Experimental Results

Under experimental conditions specified for SBD, impacted rats were behaviorally evaluated with time. Results were based on summation scores of circling response when rats were dangled head-down by holding the tip of tail (severe: 2; mild: 1; normal: 0) and forelimb catatonia (severe: 2; mild: 1; normal: 0) revealed significant abnormalities in behavioral responses. The behavioral abnormalities recovered within 12 hours post-impact.

MRI portrayals revealed significant differences in post-impact changes of cerebral injuries between non-treated and MK-801-treated animals at 48 hours after impact. T2-weighted high-signal and T1-weighted low-signal areas were manifested extensively in superficial regions and subcortical white matter of non-treated group with similar intensity/extent. Furthermore edematous development in proportion to necrosis-like tissue damage were established in the non-treated group as well.

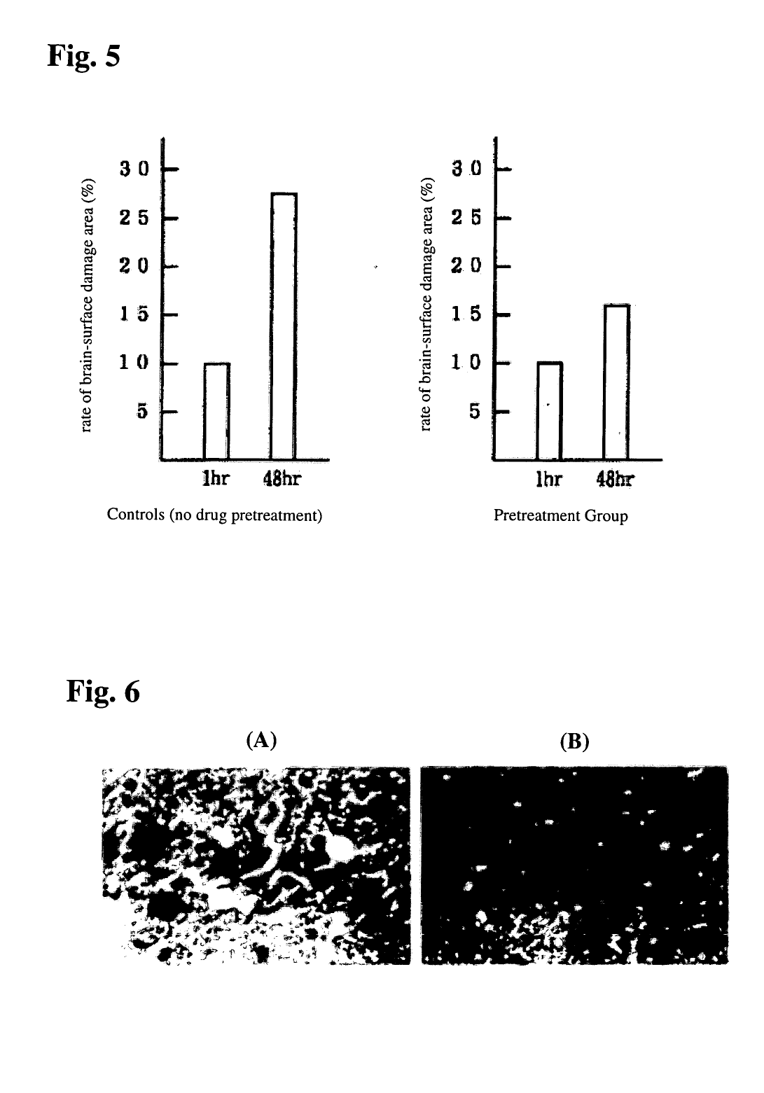

Compared with findings of non-treated animals, tissue damage areas along with edematous development and portrayals of cerebral inflict were markedly attenuated in MK-801-treated rats at 48 hr post-impact (FIG. 5a, 5b; T2-weighted MRI findings at 48 hr post-impact), although T2-weighted high-signal and T1-weighted low-signal areas at 1 hr post-impact were expanded.

In histopathological studies, coronal sections revealed brain damage appeared immediately ventral to the impact-site on the brain surface, the damage was especially marked at 48 hr post-impact. Although tissue injuries between the MK-801 and non-treated groups were different by about 10%, which appeared insignificant in damage areas, significant difference in brain damage was established at 48 hr post-impact. In the non-treated group, the damage area increased from 10% to 27.7% at 1 and 48 hr post-impact, indicating an exacerbation of 17.7% in damage. Compared with this damage, the rate was a mere 6.7% exacerbation in the MK-801-treated group (FIG. 5). In the drug-treated group, although the damage area was increased at 1 hr, the expansion of tissue damage area at 48 hr post-impact was significantly inhibited compared with non-treatment. Histopathological findings of SBD (hippocampal CA3 field) at 48 hr post-impact are illustrated for the non-treated (FIG. 6a) and treated (FIG. 6b) groups.

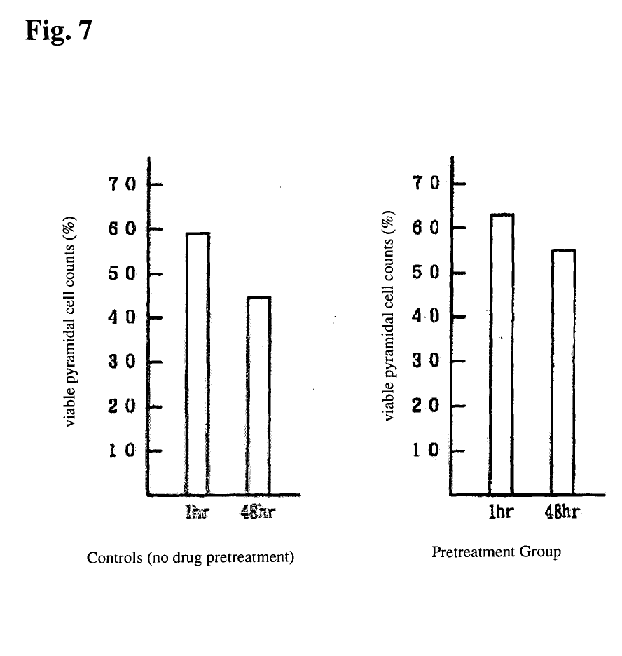

Viable cell counts per unit area for pyramid cells at the hippocampal CA1 field of the non-treated group indicated decreases of 59.2 and 44.5% at 1 and 48 (FIG. 7a) hr post-impact compared with the control group. However, the respective decreases for the pretreated group were 66.4 and 54.8% at 1 and 48 hr post-impact (FIG. 7b).

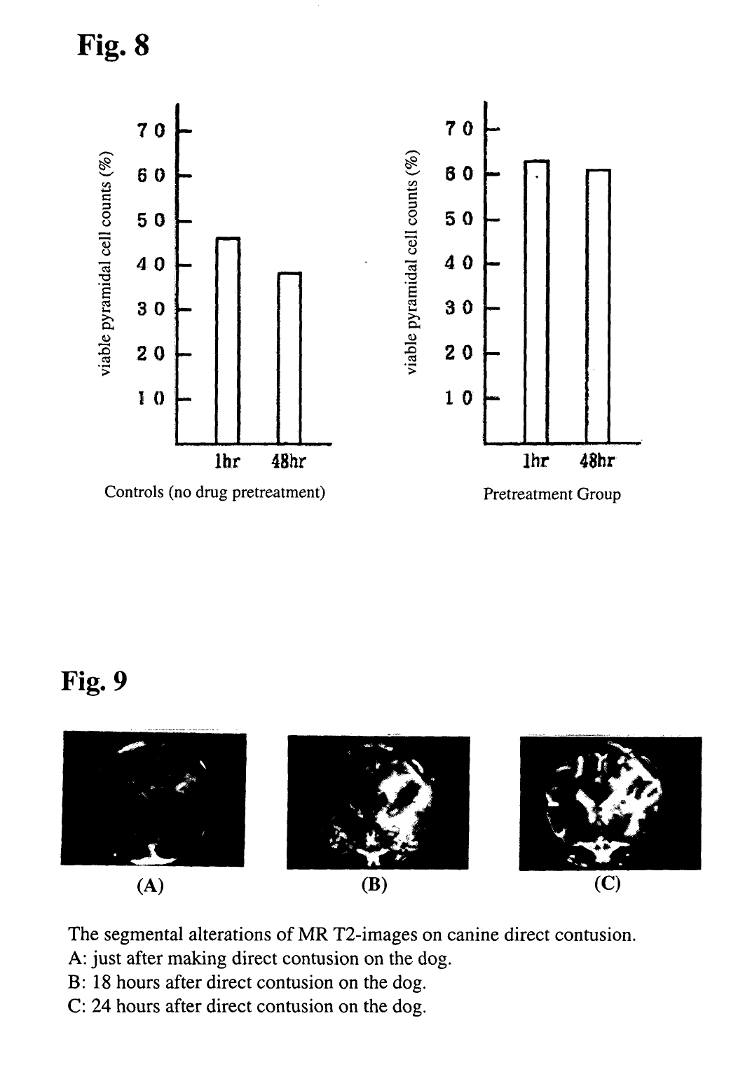

Similar investigations in the hippocampal CA3 field revealed decreases of 45.9 and 37.6% (FIG. 8a), while the rates were 63.0 and 61.2% (FIG. 8b) at 1 and 48 hr post-impact, respectively.

The above findings demonstrated that potential cytoprotective effects were established at CA1 and CA3 fields in the hippocampus: the favorable effects were especially obtained at 48 hr (cf. 1 hr) post-impact. In addition both the damage area and viable cell count were markedly attenuated in the drug-treated group at 48 hr post-impact compared with the non-treated group, implying that cytoprotective effects were established.

Although increases in viable cell counts were achieved in the treated group, the counts were less at 48 hr compared with that at 1 hr post-impact, suggesting that MK-801 could not completely suppress SBD exacerbation. Our findings demonstrated that the drug administration was effective against post-traumatic cerebral injuries, especially useful in the suppression of severe SBD expansion.

### Experiment 3

Although the clinical significance of SBD is critical for favorable treatment, experimental SBD has been attempted due to unavailability of a reliable approach. In the present study, effects of the present invention on cerebral blood flow (CBF) rate (cm/sec) in dogs, employed as an index of SBD, were investigated. Comparative studies of SBD generated with the present drop weight apparatus and method and conventional direct contusion nest (DCN) were performed.

Experimental Methods

Eight healthy 4-year-old male Beagle dogs were anesthetized before a square piece of skull (1.2×1.2 cm²) was craniectomized. As two different cerebral injuries were involved in the study, a unilateral parietal bone was drilled and the brain surface surgically exposed without removing the dura mater. In two randomized dogs, a self-designed 5-mm spherical DCN with reproducibility was applied to generate concussion (CC in Table 1). In the remaining four dogs, the WD method was employed under the following experimental conditions: a weight of 12-g mass with a drop-distance of 20 cm to generate an impact of 240 cm·g.

MRI Findings and CBF Velocity Determination

MRI findings were performed with superconductor magnetism of magnetic field intensity 0.5T (Flexart MRT-50GP; Toshiba Medical Co., Ltd.). CBF velocities of DCN (n=2) and drop-weight-inflicted (n=4) dogs (including craniectomized skull-replaced dogs) were measured via MRI using the 3D-phase shift magnetic resonance angiography with presaturation pulse (PAMRA: TE=25, TR=10). Craniectomized skull-chips were replaced and kept in position with adhesive.

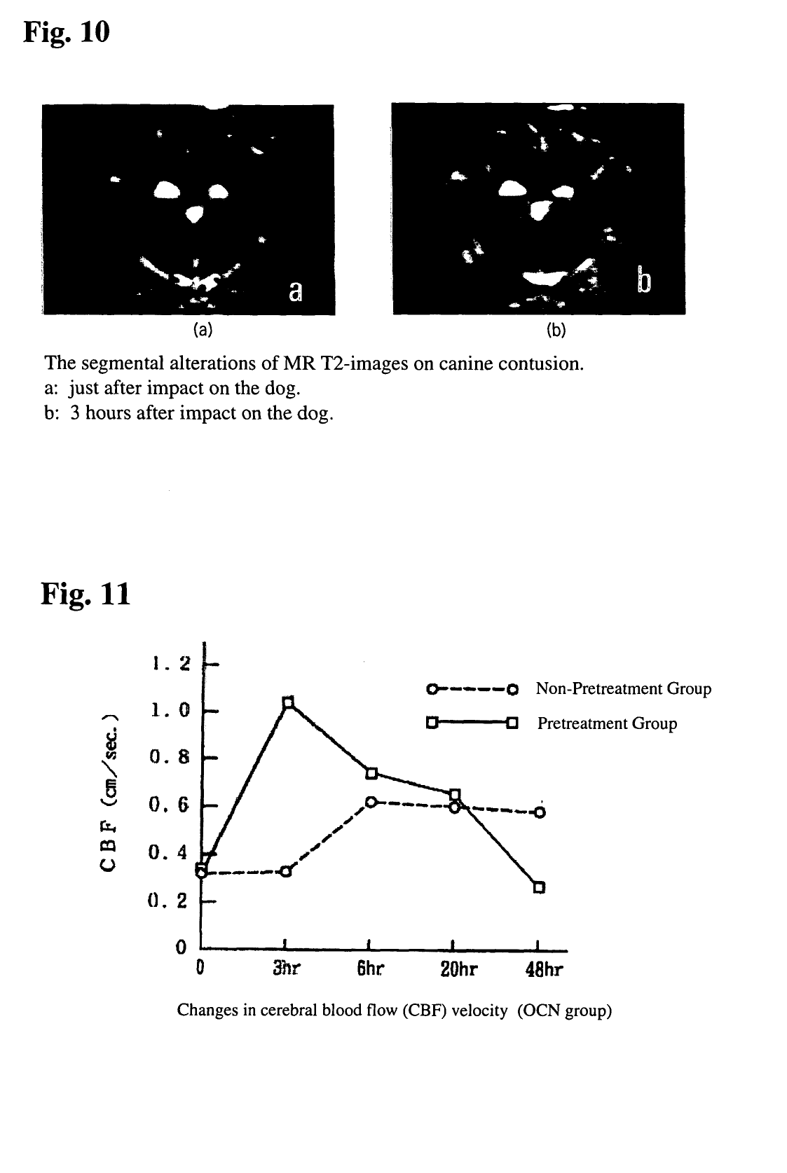

According to MRI findings in the DCN group with drug pretreatment, a damage field of 5-mm diameter with high-intensity signal (HIS) was indicated immediately after impact (time 0). The HIS field gradually changed to low-intensity signal (LIS) field with time, albeit the HIS field was observed peripheral to the LIS field, illustrating that the damage area had expanded. From these data, edema and ischemia had developed in vicinities surrounding the DCN focus. However, in the drop weight group with drug pretreatment, MRI findings immediately after impact (time 0) indicated meager spots with HIS field and subsequent time-related expansion of damage was not established (FIG. 10). This implies that SBD development was induced 3 hr after impact with the specified condition of 240 cm·g. In addition, MRI findings between the DCN and drop weight groups portrayed different patterns of damage: the former indicated a higher tendency of dispersed development of ischemia and edema compared with the latter. In other words, incidence of SBD eventuated from drop weight impact, while such is not the case with DCN where SBD is not found.

As for CBF, high values were registered immediately after impact with no subsequent changes thereafter in both non-treated (broken plot) and drug-treated (bold plot) groups subjected to DCN (FIG. 11). In short, damage to CBF in impact-inflicted sites remained irreversible without increased compensatory effects; in fact attenuated compensatory effects were established.

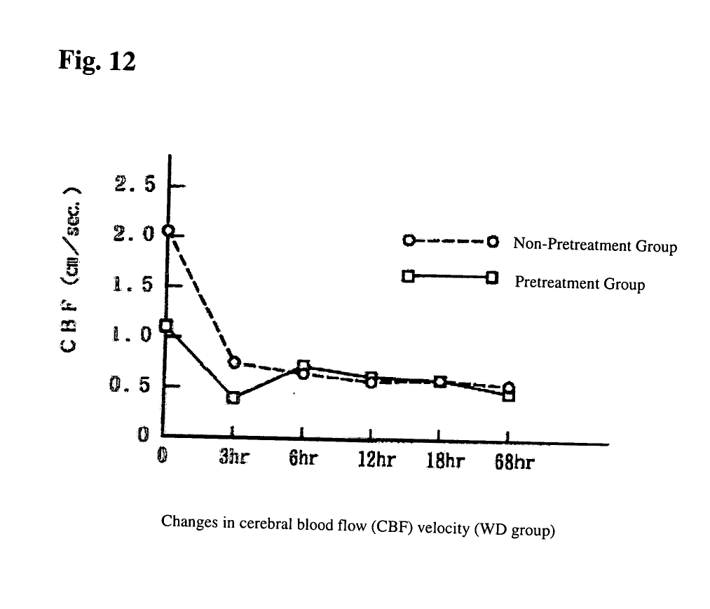

However, non-treated animals of the drop-weight-inflicted group indicated that sites where lesions were not apparent during the early phase developed into SBD with time. In addition, canine SBD was only apparent 3 hr after impact, whereupon CBF had then developed the peak value. Furthermore, time-related SBD exacerbation apparent from the MRI findings was induced with decreasing CBF (FIG. 12). Moreover, in the drop-weight-inflicted group with replaced skull-chips, changes in CBF were not evident (data not shown), implying CBF was independent of punctured or non-punctured skull. In this aspect, the peak where SBD developed was not reproducible in the drug-pretreated group, suggesting that the drug had effectively suppressed SBD development.

These results demonstrate that changes in CBF are not dependent on intracranial pressure but are markedly determined by severity of lesions inflicted. This therefore can account for the different outcomes in the DCN and weight drop methods, where the latter inflicts SBD by elevating CBF of delayed mode. As such, it is hence confirmed that the drop weight method induces CBF of delayed mode to eventuate SBD. Furthermore, time-related MRI findings can beneficially monitor and evaluate anticytotoxic effects of novel chemical agents.

### Experiment 4

The drop weight apparatus and method were used to evaluate memory using the maze. Male Fisher rats weighing 400-450 g were anesthetized with sodium pentobarbital (Nembutal, Dainippon Pharm. Co., Ltd.) by i.p. administration (40 mg/kg) before the head of animal was restrained in a stereotaxic apparatus followed by infliction of cerebral injuries using the drop weight method. Two groups of three rats each were subjected to the following conditions: one was subjected to SBD development (i.e., by dropping a weight of 5-g mass over a distance of 10 cm to generate an impact of 50 cm·g) while the other group was not (i.e., dropping a weight of 5-g mass over a distance of 1 cm to generate an impact of 5 cm·g). Using the maze, the search-reward times before and after impact on rats were measured, confirming incidences of memory impairment (Table 2). The channels of 30-cm height were of 15-cm width and extended to as far as 50 cm to the closed end of channel where a feed lot of 1-cm height with a 2.5-cm² square receptacle attached to wall of the end-side. A 20-cm space for free movement leading to the channels was allotted, and the maze measured 70 cm×62 cm on a plan view. Note that animals were allowed to learn and acquire memory on the path leading to feed (reward) by consistently placing the feed on the same feed receptacle of the channel for all rats over a period of 7 days. One day before testing, animals were not fed for 24 hours. Testing of animals was consistently performed at the same time daily during the learning process and on the test day. Use of feed was restricted to the same manufacturer, and the commercially available animal feed of 2-3-mm pellets (ca. 20 g) was repeatedly employed for each experiment.

TABLE 2
Search-reward times before and after impact on rats were measured
using the maze, confirming incidences of memory impairment in rats.

| | Search-reward* time-interval of | | |
|---|---|---|---|
| Group | Rat 1 | Rat 2 | Rat 3 |
| I (SBD** induced) | | | |
| Before learning | 29 min 05 sec | 45 min 00 sec | 8 min 03 sec |
| Pre-impact | 05 sec | 28 sec | 05 sec |
| Post-impact | 2 min 05 sec | 1 min 25 sec | 45 sec |

II (SBD** not induced)

| | | | |
|---|---|---|---|
| Before learning | 13 min 05 sec | 43 min 00 sec | 32 min 33 sec |
| Pre-impact | 23 sec | 09 sec | 06 sec |
| Post-impact | 23 sec | 09 sec | 06 sec |

*Search-reward time-interval: time taken for rats from the acclimatization room to locate the feed placed on the feed lot at the end of channels
**Secondary brain damage

Under experimental conditions for inducing SBD, the search-reward times of animals were significantly extended (Table 2), while delay was not encountered in animals impacted without SBD development, confirming that the drop weight apparatus can induce SBD. In addition, the drop weight method affected memory-related brain sites after impact. Although not indicated in the figure, MRI diagnosis before memory evaluation manifested injuries in CA1 and/or CA3 fields of animals subjected to the drop weight method.

Outcomes of Present Invention

Using the present simple and reliable experimental brain damage model, various cerebral injuries, especially SBD, can be induced reproducibly via simple experimental procedures in rats, cats and dogs using a simple apparatus. MRI diagnosis thereafter can further evaluate time-related brain damage and furnish an approach to evaluate and monitor prophylaxis and recovery of SBD that accompanied the external head injury. Furthermore, diagnosis of time-related brain injuries using memory evaluation in drop weight method-derived maze provides similar evaluation of brain damage.

The present invention, especially in discovering concrete evidence on SBD development in impacted animals can be reliable, or diagnosis immediately after imaging of MRI application may readily discriminate the absence/presence of SBD after impact. As such, the number of animals used can be reduced. Furthermore, the invention may contribute to screening of novel compounds and afford evaluation of drug efficacy of prophylactics and therapeutics for cerebral impairments, especially SBD.

1. An apparatus for inflicting brain injury in a controlled manner by dropping a weight under the force of gravity onto the exposed brain surface of an animal from a height previously determined to be appropriate to generate the desired brain injury for the animal species, said apparatus comprising a barrel defining a delivery channel extending in a vertical direction and having a terminating end for positioning adjacent the exposed brain surface of an animal and a drop weight disposed within said delivery channel.

2. An apparatus according to claim 1, further comprising: a weight restraining means for temporarily supporting said weight at a desired distance above the exposed brain surface until the user selectively removes said weight restraining means to allow the weight to fall the desired distance under the force of gravity.

3. An apparatus according to claim 2 wherein said weight retaining means includes a pin extending horizontally through diametrically opposed holes defined in said barrel.

4. An apparatus according to claim 3 wherein said barrel defines more than one set of diametrically opposed holes for supporting said pin therethrough, said sets being vertically spaced along said barrel.

5. An apparatus according to claim 1, wherein said barrel is open at both ends.

6. The apparatus of claim 1, wherein said distance is selected to induce concussion, contusion or secondary brain damage.

7. A kit for inflicting brain injury in a controlled manner by dropping a weight under the force of gravity onto the exposed brain surface of an animal from a height previously determined to be appropriate to generate the desired brain injury for the animal species and for time-related evaluation of chemical agents on memory or memory-related events, said kit comprising:
a) a drop weight apparatus having a barrel defining a delivery channel extending in a vertical direction and having a terminating end for positioning adjacent the exposed brain surface of an animal and a drop weight disposed within said delivery channel; and
b) a maze having a first open space connected to a second open space, said second open space being in communication with at least four paths with food at the end of one path, said maze allowing free horizontal movement of the animal between the open spaces and the four paths.

8. A method of inducing brain injury in a controlled manner comprising the steps of:
a) removing a portion of the skull of an animal to expose a portion of its brain with its dura mater intact;
b) providing a barrel defining a delivery channel terminating adjacent the exposed brain surface of the animal and positioning said delivery channel to extend vertically;
c) providing a drop weight within said delivery channel;
d) positioning said drop weight at a selected height above the exposed brain surface, said height being chosen based upon the species of the animal and upon the degree of brain damage to be inflicted;
e) dropping said weight under the force of gravity to impact the exposed brain surface.

9. The method of claim 16 wherein said weight is a cylindrical rod having a mass of 5 grams and a surface area at its impact end of about 19.63 mm² and wherein said weight is dropped a distance of between 5 and 25 centimeters to generate brain injury in a rat.

10. The method of claim 17 wherein said weight is dropped a distance of 10 cm to generated secondary brain damage in a rat.

11. The method of claim 18 wherein said weight is a cylindrical rod having a mass of 12 grams and a surface area at its impact end of about 19.63 mm² and wherein said weight is dropped a distance of between 5 and 25 centimeters to generate brain injury in a cat or dog.

12. The apparatus of claim 11 wherein said weight is dropped a distance of 20 cm to generated secondary brain damage in a cat or dog.

13. The method of claim 8 wherein said weight is a cylindrical rod having a mass of 15 grams and a surface area at its impact end of about 19.63 mm² and wherein said weight is dropped a distance of between 5 and 25 centimeters to generate brain injury in a cat or dog.

14. A method for inflicting brain injury in an experimental animal in a controlled manner and for time-related evaluation of chemical agents on memory or memory-related events, said method comprising the steps of:
a) providing a drop weight apparatus having a barrel defining a delivery channel extending in a vertical direction and having a terminating end for positioning adjacent the exposed brain surface of an animal and a drop weight disposed within said delivery channel; and
b) providing a maze whereby experimental animals are allowed to move freely in horizontal directions with visible planar views within a certain open space that connects to another open space with channels of at least four paths where animal feed as a reward is placed at the end of a path; and
c) prior to inflicting brain injury, training experimental animal to remember the path that leads to a reward within the shortest time-interval;
d) administering drug;
e) inflicting brain injury by:
i) removing a portion of the skull of an animal to expose a portion of its brain with its dura mater intact;
ii) positioning said delivery channel to extend vertically and to terminate adjacent the animal's exposed brain surface;
iii) positioning said drop weight at a selected height above the exposed brain surface, said height being chosen based upon the species of the animal and upon the degree of brain damage to be inflicted;
iv) dropping said weight under the force of gravity to impact the exposed brain surface;
f) monitoring time-related impact-induced cerebral injuries by timing the animals performance in the maze.

15. A method for inflicting brain injury in an experimental animal in a controlled manner and for time-related evaluation of chemical agents on memory or memory-related events, said method comprising the steps of:
a) providing a drop weight apparatus having a barrel defining a delivery channel extending in a vertical direction and having a terminating end for positioning adjacent the exposed brain surface of an animal and a drop weight disposed within said delivery channel; and
b) administering drug;
c) inflicting brain injury by:
i) removing a portion of the skull of an animal to expose a portion of its brain with its dura mater intact;
ii) positioning said delivery channel to extend vertically and to terminate adjacent the animal's exposed brain surface;
iii) positioning said drop weight at a selected height above the exposed brain surface, said height being chosen based upon the species of the animal and upon the degree of brain damage to be inflicted;
iv) dropping said weight under the force of gravity to impact the exposed brain surface;
d) monitoring time-related post-impact cerebral injuries by MRI diagnosis to evaluate drug effects of test-compounds on said brain injuries.
